(19) United States
(12) Patent Application Publication
(10) Pub. No.: US 2024/0341734 A1
(43) Pub. Date: Oct. 17, 2024

(54) Ultrasound Depth Calibration for Improving Navigational Accuracy

(71) Applicant: Medtronic Navigation, Inc., Lafayette, CO (US)

(72) Inventors: Elliot C. SCHMIDT, Minneapolis, MN (US); Brett D. JACKSON, Shoreview, MN (US); Varun A. Bhatia, Minneapolis, MN (US)

(73) Assignee: Medtronic Navigation, Inc., Lafayette, CO (US)

(21) Appl. No.: 18/630,601

(22) Filed: Apr. 9, 2024

Related U.S. Application Data
(60) Provisional application No. 63/459,153, filed on Apr. 13, 2023.

(51) Int. Cl. A61B 8/00; A61B 8/08

(52) U.S. Cl. CPC A61B 8/587; A61B 8/5207

(57) ABSTRACT

A method of calibrating an ultrasound imaging system including: capturing ultrasound image data including a first tissue and a second tissue through which ultrasound waves travel at different speeds, the ultrasound image data captured based on a predetermined single speed of ultrasound waves through both the first tissue and the second tissue being the same; segmenting the first tissue and the second tissue in a sonogram based on the image data; identifying a first depth of the first tissue and a second depth of the second tissue based on the sonogram; identifying an actual first speed of ultrasound waves through the first tissue and an actual second speed of ultrasound waves through the second tissue; and generating a calibrated image that accounts for ultrasound waves through the first tissue at the first actual speed that is different than the second actual speed of the ultrasound waves through the second tissue.

### CROSS-REFERENCE TO RELATED APPLICATION

This application claims the benefit of and priority to U.S. Provisional Patent Application No. 63/459,153 filed Apr. 13, 2023, the entire disclosure of which is incorporated by reference herein.

### FIELD

The present disclosure relates to ultrasound depth calibration for improving navigational accuracy.

### BACKGROUND

This section provides background information related to the present disclosure, which is not necessarily prior art.

Ultrasonic imaging systems are used to image various areas of a subject. The subject may include a patient, such as a human patient. The areas selected for imaging include internal areas covered by various layers of tissue and organs. To ensure accuracy, the imaging system is calibrated prior to use.

### SUMMARY

This section provides a general summary of the disclosure, and is not a comprehensive disclosure of its full scope or all of its features.

The present disclosure includes a method of calibrating an ultrasound imaging system including: capturing ultrasound image data including a first tissue and a second tissue through which ultrasound waves travel at different speeds, the ultrasound image data captured based on a predetermined single speed of ultrasound waves through both the first tissue and the second tissue being the same; segmenting the first tissue and the second tissue in a sonogram based on the image data; identifying a first depth of the first tissue and a second depth of the second tissue based on the sonogram; identifying an actual first speed of ultrasound waves through the first tissue and an actual second speed of ultrasound waves through the second tissue; and generating a calibrated image that accounts for ultrasound waves through the first tissue at the first actual speed that is different than the second actual speed of the ultrasound waves through the second tissue.

The present disclosure further includes an ultrasound imaging system having an ultrasound housing including a transducer configured to emit and receive ultrasound waves. The system further includes an image processing unit configured to: capture ultrasound image data including a first tissue and a second tissue through which ultrasound waves travel at different speeds, the ultrasound image data captured based on a predetermined single speed of ultrasound waves through both the first tissue and the second tissue being the same, wherein a sonogram is based on the ultrasound image data; segment the first tissue and the second tissue in the sonogram; identify a first depth of the first tissue and a second depth of the second tissue based on the sonogram; identify an actual first speed of ultrasound waves through the first tissue and an actual second speed of ultrasound waves through the second tissue; and generate a calibrated image based on the sonogram that accounts for ultrasound waves traveling through the first tissue at the first actual speed that is different than the second actual speed of the ultrasound waves traveling through the second tissue.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of select embodiments and not all possible implementations, and are not intended to limit the scope of the present disclosure.

FIG. 1 is an environmental view of an imaging and navigation system in accordance with the present disclosure;

FIG. 2 is a perspective view of an exemplary ultrasound housing and an ultrasound transmission plane;

FIG. 3A is an exemplary ultrasound image captured assuming uniform speed of sound through all imaged tissues;

FIG. 3B is the image of FIG. 3A revised in accordance with the present disclosure to account for sound traveling through different tissues at different speeds;

FIG. 4A is another exemplary ultrasound image captured assuming uniform speed of sound through all imaged tissues;

FIG. 4B is the image of FIG. 4A revised in accordance with the present disclosure to account for sound traveling through different tissues at different speeds; and

FIG. 5 illustrates a method in accordance with the present disclosure for ultrasound depth calibration to improve navigational accuracy.

Corresponding reference numerals indicate corresponding parts throughout the several views of the drawings.

### DETAILED DESCRIPTION

Example embodiments will now be described more fully with reference to the accompanying drawings. As discussed herein, a cine loop can refer to a plurality of images acquired at a selected rate of any portion. The plurality of images can then be viewed in sequence at a selected rate to indicate motion or movement of the portion. The portion can be an anatomical portion, such as a heart, or a non-anatomical portion, such as a moving engine or other moving system.

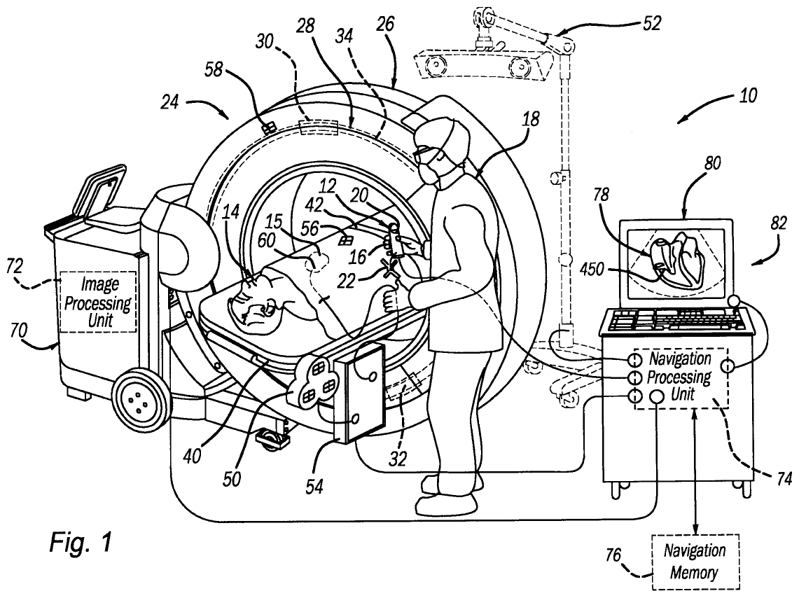

FIG. 1 is a diagram illustrating an overview of a navigation system 10 that can be used for various procedures. The navigation system 10 can be used to track the location of an item, such as an implant or an instrument, and at least one imaging system 12 relative to a subject, such as a patient 14. The navigation system 10 may be used to navigate any type of instrument, implant, or delivery system, including, but not limited to, the following: guide wires, arthroscopic systems, ablation instruments, stent placement, orthopedic implants, spinal implants, deep brain stimulation (DBS) probes, etc. Non-human or non-surgical procedures may also use the navigation system 10 to track a non-surgical or non-human intervention of the instrument or imaging device. Moreover, the instruments may be used to navigate or map any region of the body. The navigation system 10 and the various tracked items may be used in any appropriate procedure, such as one that is generally minimally invasive or an open procedure.

The navigation system 10 can interface with, or integrally include, an imaging system 12 that is used to acquire pre-operative, intra-operative, or post-operative, or real-time image data of the patient 14. For example, the imaging system 12 can be an ultrasound imaging system (as discussed further herein) that has a tracking device 22 attached thereto (i.e. to be tracked with the navigation system 10), but only provides a video feed to a navigation processing unit 72 to allow capturing and viewing of images on a display device 80. Alternatively, the imaging system 12 can be integrated into the navigation system 10, including a navigation processing unit 74.

Any appropriate subject can be imaged and any appropriate procedure may be performed relative to the subject. The navigation system 10 can be used to track various tracking devices, as discussed herein, to determine locations of the patient 14. The tracked locations of the patient 14 can be used to determine or select images for display to be used with the navigation system 10. The initial discussion, however, is directed to the navigation system 10 and the exemplary imaging system 12.

In the example shown, the imaging system includes an ultra-sound (US) imaging system 12 that includes a US housing 16 that is held by a user 18 while collecting image data of the subject 14. The US housing 16 can also be held by a stand or robotic system while collecting image data. The US housing and included transducer can be any appropriate US imaging system 12, such as the M-TURBO® sold by SonoSite, Inc. having a place of business at Bothell, Washington. Associated with, such as attached directly to or molded into, the US housing 16 or the US transducer housed within the housing 16 is at least one imaging system tracking device, such as an electromagnetic tracking device 20 and/or an optical tracking device 22. The tracking devices can be used together (e.g. to provide redundant tracking information) or separately. Also, only one of the two tracking devices may be present. It will also be understood that various other tracking devices can be associated with the US housing 16, as discussed herein, including acoustic, ultrasound, radar, and other tracking devices. Also, the tracking device can include linkages or a robotic portion that can determine a location relative to a reference frame.

FIG. 1 further illustrates a second imaging system 24, which includes an O-Arm® imaging device sold by Medtronic Navigation, Inc. having a place of business in Louisville, Colorado, USA. The second imaging device 24 includes imaging portions, such as a generally annular gantry housing 26 that encloses an image capturing portion 28. The image capturing portion 28 may include an x-ray source or emission portion 30 and an x-ray receiving or image receiving portion 32. The emission portion 30 and the image receiving portion 32 are generally spaced about 180 degrees from each other and mounted on a rotor (not illustrated) relative to a track 34 of the image capturing portion 28. The image capturing portion 28 can be operable to rotate 360 degrees during image acquisition. The image capturing portion 28 may rotate around a central point or axis, allowing image data of the patient 26 to be acquired from multiple directions or in multiple planes.

The second imaging system 24 can include those disclosed in U.S. Pat. Nos. 7,188,998; 7,108,421; 7,106,825; 7,001,045; and 6,940,941; all of which are incorporated herein by reference. The second imaging system 24 can, however, generally relate to any imaging system that is operable to capture image data regarding the subject 14 other than the US imaging system 12 or in addition to a single US imaging system 12. The second imaging system 24, for example, can include a C-arm fluoroscopic imaging system which can also be used to generate three-dimensional views of the patient 14.

The patient 14 can be fixed onto an operating table 40, but is not required to be fixed to the table 40. The table 40 can include a plurality of straps 42. The straps 42 can be secured around the patient 14 to fix the patient 14 relative to the table 40. Various apparatuses may be used to position the patient 40 in a static position on the operating table 40. Examples of such patient positioning devices are set forth in commonly assigned U.S. patent application Ser. No. 10/405,068, published as U.S. Pat. App. Pub. No. 2004-0199072 on Oct. 7, 2004, entitled “An Integrated Electromagnetic Navigation And Patient Positioning Device”, filed Apr. 1, 2003 which is hereby incorporated by reference. Other known apparatuses may include a Mayfield® clamp.

The navigation system 10 includes at least one tracking system. The tracking system can include at least one localizer. In one example, the tracking system can include an EM localizer 50. The tracking system can be used to track instruments relative to the patient 14 or within a navigation space. The navigation system 10 can use image data from the imaging system 12 and information from the tracking system to illustrate locations of the tracked instruments, as discussed herein. The tracking system can also include a plurality of types of tracking systems including an optical localizer 52 in addition to and/or in place of the EM localizer 50. When the EM localizer 50 is used, the EM localizer can communicate with or through an EM controller 54. Communication with the EM controller can be wired or wireless.

The optical tracking localizer 52 and the EM localizer 50 can be used together to track multiple instruments or used together to redundantly track the same instrument. Various tracking devices, including those discussed further herein, can be tracked and the information can be used by the navigation system 10 to allow for an output system to output, such as a display device to display, a position of an item. Briefly, tracking devices, can include a patient or reference tracking device (to track the patient 14) 56, a second imaging device tracking device 58 (to track the second imaging device 24), and an instrument tracking device 60 (to track an instrument 62), allow selected portions of the operating theater to be tracked relative to one another with the appropriate tracking system, including the optical localizer 52 and/or the EM localizer 50. The reference tracking device 56 can be positioned on the instrument 62 (e.g. a catheter) to be positioned within the patient 14, such as within a heart 15 of the patient 14.

It will be understood that any of the tracking devices 20, 22, 56, 58, 60 can be optical or EM tracking devices, or both, depending upon the tracking localizer used to track the respective tracking devices. It will be further understood that any appropriate tracking system can be used with the navigation system 10. Alterative tracking systems can include radar tracking systems, acoustic tracking systems, ultrasound tracking systems, and the like. Each of the different tracking systems can be respective different tracking devices and localizers operable with the respective tracking modalities. Also, the different tracking modalities can be used simultaneously as long as they do not interfere with each other (e.g. an opaque member blocks a camera view of the optical localizer 52).

An exemplary EM tracking system can include the STEALTHSTATION® AXIEM™ Navigation System, sold by Medtronic Navigation, Inc. having a place of business in Louisville, Colorado. Exemplary tracking systems are also disclosed in U.S. Pat. No. 7,751,865, issued Jul. 6, 2010 and entitled “METHOD AND APPARATUS FOR SURGICAL NAVIGATION”; U.S. Pat. No. 5,913,820, titled “Position Location System,” issued Jun. 22, 1999 and U.S. Pat. No. 5,592,939, titled “Method and System for Navigating a Catheter Probe,” issued Jan. 14, 1997, all herein incorporated by reference.

Further, for EM tracking systems it may be necessary to provide shielding or distortion compensation systems to shield or compensate for distortions in the EM field generated by the EM localizer 50. Exemplary shielding systems include those in U.S. Pat. No. 7,797,032, issued on Sep. 14, 2010 and U.S. Pat. No. 6,747,539, issued on Jun. 8, 2004; distortion compensation systems can include those disclosed in U.S. patent Ser. No. 10/649,214, filed on Jan. 9, 2004, published as U.S. Pat. App. Pub. No. 2004/0116803, all of which are incorporated herein by reference.

With an EM tracking system, the localizer 50 and the various tracking devices can communicate through the EM controller 54. The EM controller 54 can include various amplifiers, filters, electrical isolation, and other systems. The EM controller 54 can also control the coils of the localizer 52 to either emit or receive an EM field for tracking. A wireless communications channel, however, such as that disclosed in U.S. Pat. No. 6,474,341, entitled “Surgical Communication Power System,” issued Nov. 5, 2002, herein incorporated by reference, can be used as opposed to being coupled directly to the EM controller 54.

It will be understood that the tracking system may also be or include any appropriate tracking system, including a STEALTHSTATION® TRIA®, TREON®, and/or S7™ Navigation System having an optical localizer, similar to the optical localizer 52, sold by Medtronic Navigation, Inc. having a place of business in Louisville, Colorado. Further, alternative tracking systems are disclosed in U.S. Pat. No. 5,983,126, to Wittkampf et al. titled “Catheter Location System and Method,” issued Nov. 9, 1999, which is hereby incorporated by reference. Other tracking systems include an acoustic, radiation, radar, etc. tracking or navigation systems.

The second imaging system 24 can further include a support housing or cart 70 that can house the image processing unit 72. The cart 70 can be connected to the gantry 26. The navigation system 10 can include a navigation processing unit 74 that can communicate or include a navigation memory 76. The navigation processing unit 74 can include a processor (e.g. a computer processor) that executes instructions to determine locations of the tracking devices based on signals from the tracking devices. The navigation processing unit 74 can receive information, including image data, from the imaging system 12 and/or the second imaging system 24 and tracking information from the tracking systems, including the respective tracking devices and/or the localizers 50, 54. Image data can be displayed as an image 78 on a display device 80 of a workstation or other computer system 82 (e.g. laptop, desktop, tablet computer which may have a central processor to act as the navigation processing unit 74 by executing instructions). The workstation 82 can include appropriate input devices, such as a keyboard 84. It will be understood that other appropriate input devices can be included, such as a mouse, a foot pedal or the like which can be used separately or in combination. Also, all of the disclosed processing units or systems can be a single processor (e.g. a single central processing chip) that can execute different instructions to perform different tasks.

The image processing unit 72 processes image data from the second imaging system 24 and a separate first image processor (not illustrated) can be provided to process or pre-process image data from the imaging system 12. The image data from the image processor can then be transmitted to the navigation processor 74. It will be understood, however, that the imaging systems need not perform any image processing and the image data can be transmitted directly to the navigation processing unit 74. Accordingly, the navigation system 10 may include or operate with a single or multiple processing centers or units that can access single or multiple memory systems based upon system design.

In various embodiments, the imaging system 12 can generate image data that defines an image space that can be registered to the patient space or navigation space. In various embodiments, the position of the patient 14 relative to the imaging system 12 can be determined by the navigation system 10 with the patient tracking device 56 and the imaging system tracking device(s) 20, 22 to assist in registration. Accordingly, the position of the patient 14 relative to the imaging system 12 can be determined.

Manual or automatic registration can occur by matching fiducial points in image data with fiducial points on the patient 14. Registration of image space to patient space allows for the generation of a translation map between the patient space and the image space. According to various embodiments, registration can occur by determining points that are substantially identical in the image space and the patient space. The identical points can include anatomical fiducial points or implanted fiducial points. Exemplary registration techniques are disclosed in Ser. No. 12/400,273, filed on Mar. 9, 2009, now published as U.S. Pat. App. Pub. No. 2010/0228117, which are incorporated herein by reference.

Once registered, the navigation system 10 with or including the imaging system 12, can be used to perform selected procedures. Selected procedures can use the image data generated or acquired with the imaging system 12. Further, the imaging system 12 can be used to acquire image data at different times relative to a procedure. As discussed herein, image data can be acquired of the patient 14 prior to the procedure for collection of automatically registered image data or cine loop image data. Also, the imaging system 12 can be used to acquire images for confirmation of a portion of the procedure.

In addition to registering the subject space to the image space, however, the imaging plane of the US imaging system 12 can also be determined. By registering the image plane of the US imaging system 12, imaged portions can be located within the patient 14. For example, when the image plane is calibrated to the tracking device(s) 20, 22 associated with the US housing 16 then a position of an imaged portion of the heart 15, or other imaged portion, can also be tracked.

### Calibration System

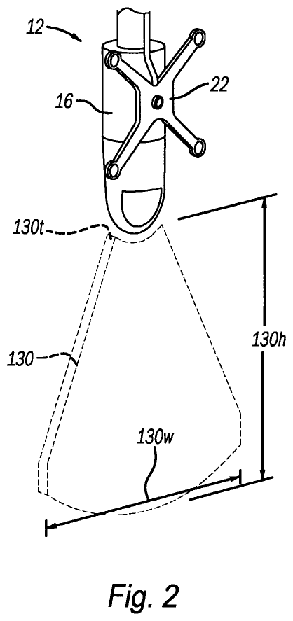

With continued reference to FIG. 1 and additional reference to FIG. 2, the ultrasound imaging system 12 emits and receives ultrasound transmissions with an ultrasound transducer (not illustrated). The US transducer can be placed in a fixed manner within the US housing 16, again as understood in the art. The US transmissions may be understood to be a specific frequency of sound or sound waves. The waves are emitted, reflect off a surface, and the reflected wave is received at a receiver. A US transducer emits and receives US waves (also referred to as sound waves). The waves travel through or propagate through a medium at a speed based at least in part on the parameters or properties of the medium. The medium may be tissue, such as human tissue.

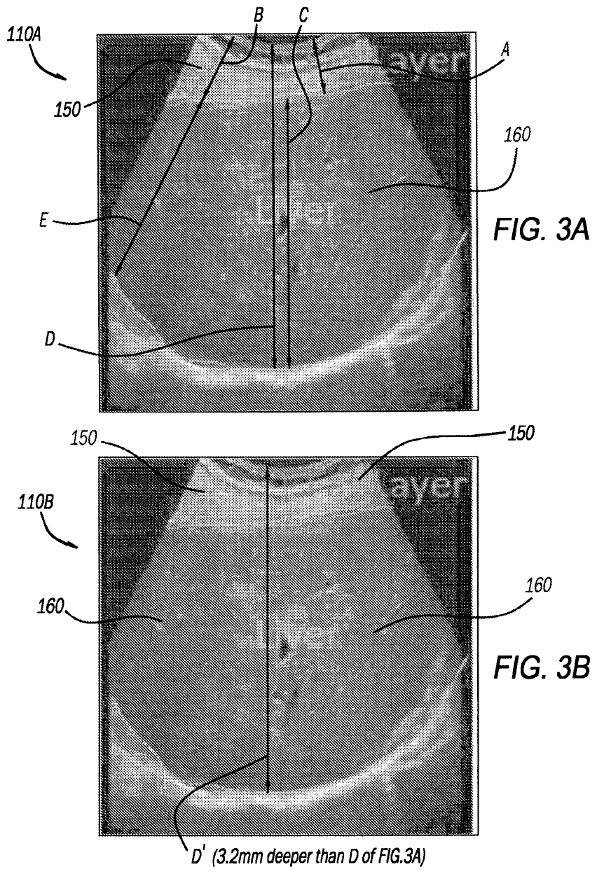

The US transmissions are generally within a plane 130 that defines a plane height 130h and a plane width 130w. The height 130h and width 130w are dimensions of the US imaging plane 130 that extend from the US housing 16. The US plane 130 can also have a thickness 130t that is negligible for calibration purposes. Generally, the ultrasound plane 130 extends from the US housing 16 at a position relative to the US housing 16 for the height 130h and the width 130w. The plane 130 can extend generally aligned with the US transducer. An image acquired within the US plane 130 can appear as illustrated in FIG. 3A at 110A.

The position of the US plane 130 is calibrated relative to the US housing 16 and various tracking devices, such as the EM tracking device 20 or the optical tracking device 22, positioned on or in the US housing 16. Once calibrated, the US imaging system 12 is a calibrated imaging system that can be used in further procedures to identify locations of imaged portions relative to the US housing 16 or the tracking devices associated with the US housing 16. For example, the US plane 130 of the calibrated imaging system 12 can be used to image portions of the subject, such as the heart 15 of the patient 14, wherein the heart wall or valve may be an imaged portion.

Once calibrated, the US housing 16 including the US tracking device 22 can be used to identify locations of imaged portions within an image acquired with the US plane 130. As discussed above, the imaged portions can include tissue, bones, or walls of the heart 15. Accordingly, when an image is acquired with the US imaging system 12, a location of an imaged portion within the US plane 130 can be determined with the navigation system 10 based upon the calibrated US plane 130 relative to the US tracking device 22. This calibration may be performed in any suitable manner. Exemplary methods and systems for performing the calibration are described in, for example, U.S. Pat. No. 8,320,653 (issued Nov. 27, 2012) and U.S. Pat. No. 9,138,204 (issued Sep. 22, 2015), which are incorporated herein in their entirety.

Sound waves generated by the transducer are reflected back to the transducer by boundaries between various tissues in the path of the beam. The ultrasound imaging system 12 performs distance measurements to synthesize images from returning echoes. To generate images for an ultrasound scan, the system 12 determines the distance of reflective interfaces from the transducer. To do so, the following formula is used: distance=(speed×time)/2; where distance is the distance between the transducer and the reflective interface; speed is propagation speed of sound waves through tissue; and time is the time taken for the pulsed sound-wave to reach the interface and the resultant echo to return to the transducer. The calculation is divided by two because the time measurement refers to the round trip of the pulsed sound wave/returning echo. An accurate measurement of the distance between the transducer and the reflective interface is thus important to achieving an accurate sonogram.

In accordance with the present disclosure, further calibration is performed to account for different tissue densities captured within the US plane 130. Traditional ultrasonic systems operate on the assumption that sound propagates through the imaged tissues uniformly at a velocity of 1540 m/s. But, the average speed of sound along any given trajectory varies through different tissues. The difference between the actual distance to the reflective interface and the distance estimated using an average velocity of 1540 m/s through all tissues can be significant for deep imaging in tissues through which sound propagates at speeds different than 1540 m/s, such as with respect to fat tissue. In the diagnostic space, this difference can lead to inaccuracies in size measurements. In the navigation space, this difference can compound with errors in positional location of tools. In a system like Emprint SX, the tool positions are localized by EM. The anatomy position is localized by first localizing the US housing 16 in the EM space, and then tying the ultrasound image to the position of the US housing 16 in the EM space (as described in, for example, U.S. Pat. No. 8,320,653 (issued Nov. 27, 2012) and U.S. Pat. No. 9,138,204 (issued Sep. 22, 2015), which are incorporated herein in their entirety). Any distance errors in the ultrasound image will then be propagated to the EM localization of the anatomical data. This may lead to misalignment errors between tool position and anatomy alignment.

The present disclosure resolves such potential misalignment issues by segmenting the different tissues imaged within the US plane 130, and calibrating the imaging system 12 and the navigation system 10 to account for the different speeds at which ultrasonic waves travel through different tissues have different tissue densities. For example, FIG. 3A illustrates an ultrasonic image 110A captured within the US plane 130 including a fat layer 150 and a liver layer 160, which have been segmented. Although only two layers have been segmented in the image 110A, any suitable number of additional layers may be segmented based on the area captured within the US plane 130. For example, a blood layer and a muscle layer may also be captured and segmented.

The present disclosure provides for segmenting and identifying the different layers in the ultrasonic image 110A in a variety of different ways. Generally, segmentation may include identification of a boundary and/or geometry of at least one object. As discussed herein, segmentation may include identifying boundaries of tissue types, (e.g., adipose tissue and organ tissue (e.g., liver)). In addition to segmentation, the type of tissue within each segmented portion may be identified. For example, a segmentation process may segment a boundary, such as by pixel contrast analysis. The identification includes determining the nature or type of tissue on either side of the boundary. As discussed herein, the identification of tissue may be used to evaluate a true ultrasound propagation speed therein.

As a first example, the segmentation and identification may be performed manually based on a visual inspection of the appearance (e.g., textures) of different tissues within the image 110A captured using 1540 m/s as the average velocity of sound through all tissue. More specifically, a person with knowledge in analyzing US images (also referred to as sonogram) will view the different tissue textures imaged using 1540 m/s as the average US velocity, such as on the display device 80 or a printout of the image. The textures may refer to pixel or image element intensity, contrast, or other visual features. The texture may also refer to as US data that may be analyzed by a system. With respect to the image 110A of FIG. 3A, the person will be able to identify the differences in appearance between the layer 150 and the layer 160, and determine based on the appearance that the layer 150 is a fat layer and the layer 160 is a liver layer and the boundaries of these layers. For example, a first region (e.g., fat) may have a first texture that is visually, or otherwise, distinguishable from a second region (e.g., liver)

As a second example, the segmentation and identification may be performed based on the physical location of the US housing 16 relative to the area being scanned. More specifically, a person knowledgeable in the area being imaged, such as human anatomy, will view the different tissues of the image 110A captured using 1540 m/s as the average US velocity, such as on the display device 80 or a printout of the image. With respect to the ultrasonic image 110A of FIG. 3A, the person will be able to identify the layer 160 as a liver layer based on the physical location of the US housing 16. Knowing that the liver layer 160 is typically below a fat layer, the knowledgeable person will be able to identify that the layer 150 is a fat layer. For example, an operator would enter into the system a predicted value (average over a population, average based on a subset population such as one of a similar race, gender, height, and weight) or by looking at the actual ultrasound image where the fat layer would be visible. When looking at that image, the operator can either enter in an average fat thickness according to what they are seeing or they can manually trace, such as on a display device, to segment the fat layer thickness in the image. The position of the fat depth can then be saved relative to navigation space (such as based on one or more of the tracking systems).

As a third example, the segmentation and identification may be carried out automatically based on an algorithm configured to analyze the texture of fat tissue, liver tissue, muscle tissue, blood etc. The algorithm may be run by the image processing unit 72, or any suitable processing module. More specifically, the algorithm is configured to analyze the different tissue textures imaged within the US plane 130. With respect to the image 110A of FIG. 3A, the algorithm is configured to identify the differences in texture between the layer 150 and the layer 160 and determine, based on the textures, the layer 150 is a fat layer and the layer 160 is a liver layer. The algorithm may also take into account the position of the US housing 16 relative to the area being scanned and the type of tissue expected to be in the area being scanned. An automatic segmentation algorithm can be trained using a supervised machine learning (ML) approach. Training, according to various embodiments, may include ultrasound images containing the tissue of interest (e.g. liver, fat, etc.) are then collected. These images are annotated with the target segmentation masks for each tissue type. Finally, a ML model (such as a convolutional neural network or vision transformer model) is trained to predict which pixels in the image (if any) correspond to that tissue type. The ML training methodology may be similar to the approaches presented in the following references, which are incorporated herein by reference: U-Net: Convolutional Networks for Biomedical Image Segmentation by Olaf Ronneberger, Philipp Fischer, and Thomas Brox, Computer Science Department and BIOSS Centre for Biological Signaling Studies, University of Freiburg, Germany (May 18, 2015); and UNETR: Transformers for 3D Medical Image Segmentation by Ali Hatamizadeh, Yucheng Tang, Vishwesh Nath, Dong Yang, Andriy Myronenko, Bennett Landman, Holger R. Roth, and Daguang Xu (Oct. 9, 2021).

After the different tissues are typed or identified and their boundaries determined based on the segmentation in the US plane 130, the geometry, include at least a depth or extent along an axis of the US plane of the different tissue segments are measured. The measurements may be performed, for example, manually based on the image (such as the image 110A) displayed on the display device 80 or based on a printout of the image of the US plane 130. Alternatively, the depth of the different tissue segments may be performed automatically by any suitable algorithm run on the image processing unit 72 or any other suitable control module. The segmenting may also be performed manually by a user segmenting the image visually rather than by an algorithm, as discussed above.

Another alternative of the present disclosure for measuring the depth of the different tissue segments includes estimating the depth based on patient parameters. For example, and with respect to the fat layer 150, the thickness of the fat layer 150 may be estimated based on one or more of the following patient parameters: body mass index (BMI); weight; age; sex, etc. The parameters are entered into the image processing unit 72 through any suitable user interface, and based on the parameters the image processing unit, or any other suitable control module, estimates the thickness of the fat layer 150. For example, if the patient has a relatively high BMI and a relatively high body weight, the thickness of the fat layer 150 will be estimated to be relatively thicker than if the patient has a relatively low BMI and a relatively low body weight. Specific thickness values assigned may be based on a lookup table with the average fat layer thicknesses of persons with various BMI's, body weights, ages, etc. for a cross-section of individuals. The thickness of the liver layer 160 may also be estimated based on a lookup table with representative liver thicknesses for individuals of various different BMI's, weights, ages, sex, etc.

Various different depth measurements may be taken into account for tissue layers having varying thicknesses. For example, and as illustrated in FIG. 3A, a first depth measurement through the fat layer 150 may be taken along line A, and a second depth measurement through the fat layer 150 may be taken along line B. The fat layer 150 is relative thinner along line A as compared to line B, so the US waves will travel a shorter distance along line A as compared to line B. Similarly, and with respect to the liver layer 160, a first depth measurement through the liver layer 160 may be taken along line C, and a second depth measurement through the liver layer 160 may be taken along line E. The liver layer 160 is relatively thicker along line C as compared to line E, so the US waves will travel a longer distance along line C as compared to line E. Any suitable number of distance measurements may be taken to account for the varying thicknesses of the tissue layers. For example, thicknesses measurements across the entire interface areas between the fat layer 150 and the liver layer 160 may be taken, and thicknesses measurements may be taken across the entire interface area of the deepest portion of the liver layer 160. The measurements may be taken manually or by algorithmically segmenting ultrasound images collected at a variety of locations. The position of those images (and fat thickness) would therefore be tied to navigation or patient space and patient anatomy.

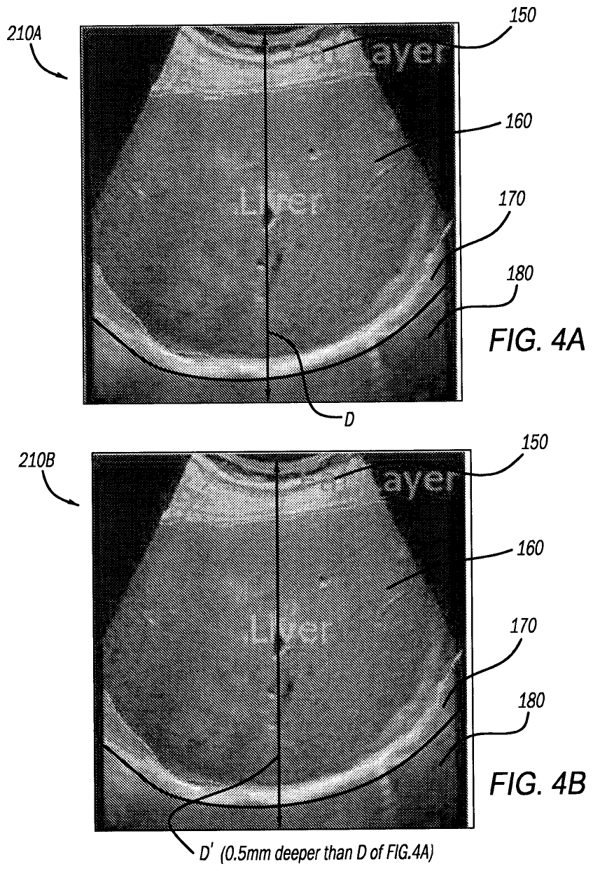

The segmentation and tissue depth measurements described above may be taken for each US image “slice” captured in the US plane 130, such as the image slices of FIGS. 3A and 4A. A calibration map may also be created by tracking the US housing 16 by EM as the US housing 16 scans over a region of interest. Various calibration zones on the map will be created for different areas of tissue density. The map will be saved at the image processing unit 72, or at any other suitable control module. Based on the type and thicknesses of the different tissue layers of the image 110A (and 210A described herein) captured in the US plane 130, the image processing unit 72 (or any other suitable control module) is configured to calibrate the imaging system 12 to account for the different or varying speeds at which ultrasonic waves travel through the different identified tissues, as set forth in the following examples.

A first depth calibration example related to percutaneous liver ablation in an obese patient will now be described. FIG. 3A illustrates the ultrasonic image 110A captured within the US plane 130 using 1540 m/s as the average speed of sound through all tissues. In accordance with the present disclosure, the image 110A is segmented into the fat layer 150 and the liver layer 160 using one or more of the exemplary segmentation processes described above. The fat layer 150 was measured to have, or estimated to have, a depth of 6 mm (0.006 m). The liver layer 160 was measured to have, or estimated to have, a depth of 10 mm (0.010 m). Thus, the image 110A of FIG. 3A is to a depth of 16 mm (0.016 m). The depth measurements may be taken at the thickest portions of the fat layer 150 and the liver layer 160 respectively, or averages of a plurality of depth measurements may be taken of the fat layer 150 and the liver 160 respectively.

Ultrasonic waves are known to travel through the segmented tissues at the following speeds: fat at 1450 m/s; and liver at 1550 m/s. Traditional ultrasound methods assume an average speed of 1540 m/s through all tissue. Thus, using traditional methods, ultrasonic waves from the US housing 16 are determined to take 10.39 microseconds to reach the 0.016 m depth of the ultrasonic image 110A: (0.016 m/1540 m/s)=10.39 microseconds. But such traditional methods fail to take into account the different speeds that ultrasonic waves travel through the different tissues such as the fat and the liver.

In accordance with the present disclosure, the actual time required for the ultrasonic waves to reach the 0.016 m depth of the image 110A, taking into account the different speeds at which sound travels through the different tissues, is as follows: (0.006 m/1450 m/s)+ (0.01 m/1550 m/s)=10.59 microseconds. Thus, there is a 2% difference between the actual maximum depth calculated in accordance with the present disclosure and the maximum depth calculated using the velocity of 1540 m/s for all tissue: 10.59/10.39=1.02; 1.02*160 mm=163.2 mm=3.2 mm error. Based on this difference, the image processing unit 72, or any other suitable control module, is configured to modify the image of FIG. 3A to increase the accuracy thereof. Alternatively, the processing unit 72 is configured to generate an initial image that is rendered using the actual speed of ultrasonic waves through known areas of tissue based on estimated or actual tissue thicknesses. In other words, the image generated is based on the segmentation and identification of tissues and their respective thicknesses and/or depths relative to the US probe. Thus, the image generated is based on actual US propagation rates in the different tissues.

FIG. 3B is an example of a reconfigured, calibrated or updated ultrasonic image 110B based on image 110A. Image 110B is reconfigured to take into account the different speeds that sound travels through the different tissues. For example, the maximum depth D of image 110A (160 mm) has been corrected to depth D′ (163.2 mm) in image 110B to correct the 3.2 mm error described above. In other words, the maximum depth D of the image 110A has been corrected to depth D′, which is 3.2 mm deeper than depth D. Various other portions of the image 110B are also corrected to take into account the different speeds that ultrasonic waves travel through fat 150 and liver 160 tissues. The image 110B is used by the navigation processing unit 74 to track instruments relative thereto. The image 110B (and likewise the image 210B) is a new image computed by scaling the original image (110A or 210A) according to a new composite speed of sound. The scaling factor will depend on the thickness of the different tissue types (liver, fat, muscle, etc.), and the known speeds of sound through those tissue types. Essentially, the pixels of the original image (110A, 210A) are repositioned based on how deep the pixels should have been, relative to the US probe or surface of the subject, using a more accurate composite speed of sound.

A second depth calibration example in accordance with the present disclosure related to a subcostal image, deep cardiac, will now be described. With reference to FIG. 4A, the image 210A was captured using 1540 m/s as the average speed of sound through all tissue. The image 210A was segmented into a fat layer 150, a liver layer 160, a muscle layer 170, and a blood layer 180 in accordance with one or more of the segmentation processes described above. The fat layer 150 was measured to have, or estimated to have, a depth of 3 mm (0.003 m). The liver layer 160 was measured to have, or estimated to have, a depth of 5 mm (0.005 m). The muscle layer 170 was measured to have, or estimated to have, a depth of 3 mm (0.003 m) based upon the segmentation. The blood layer 180 was measured to have, or estimated to have, a depth of 7 mm (0.007 m). Thus, the image 210A captured within the US plane 130 of FIG. 4A is to a depth of 18 mm (0.018 m).

Ultrasonic waves are known to travel through the segmented tissues of FIG. 4A at the following speeds: fat at 1450 m/s; liver at 1550 m/s; muscle at 1580 m/s; and blood at 1570 m/s. Traditional ultrasound methods assume an average speed of 1540 m/s through all tissue. Thus, using traditional calibration methods, the ultrasonic waves from the US housing 16 take 11.69 microseconds to reach the 0.018 m depth of the US plane 130: (0.018 m/1540 m/s)−11.69 microseconds. But such traditional methods fail to take into account the different speeds that ultrasonic waves travel through the fat, liver, muscle, and blood tissues.

In accordance with the present disclosure, the actual time required for the ultrasonic waves to reach the 0.018 m depth of the US plane 130, taking into account the different speeds as which sound travels through the different tissues, is as follows: (0.003 m/1450 m/s)+(0.005 m/1550 m/s)+(0.003 m/1580 m/s)+(0.007/1570 m/s)=11.65 microseconds. Thus, there is a 0.3% difference between the actual max depth calculated in accordance with the present disclosure and the max depth calculated using the velocity of 1540 m/s for all tissue: 11.65/11.69=0.997; 0.997*180 mm=179.5 mm=0.5 mm error. Based on this difference, the image processing unit 72, or any other suitable control module, is configured to modify the image of FIG. 4A to increase the accuracy thereof. Alternatively, the processing unit 72 is configured to generate an initial image that is rendered using the actual speed of ultrasonic waves through known areas of tissue based on estimated or actual tissue thicknesses. Thus, in accordance with the present disclosure, knowing the actual depth of the US plane 130 improves navigational accuracy of an instrument or tool relative to the anatomy.

FIG. 4B is an example of a reconfigured or updated and computed ultrasonic image 210B based on the image 210A. The image 210B is reconfigured to take into account the different speeds that sound travels through the different tissues. For example, the maximum depth D of image 210A (180 mm) has been corrected to depth D′ (179.5 mm) in image 210B to correct the 0.5 mm error described above. In other words, the maximum depth D of the image 210A has been corrected to depth D′ of image 210B, which is 0.5 mm less deep than depth D. Various other portions of the image 210B may also be corrected to take into account the different speeds that ultrasonic waves travel through fat 150, liver 160, blood 170, and muscle 180 tissues. The image 210B is used by the navigation processing unit 74 to track instruments relative thereto. The image may be corrected or changed using generally known morphing techniques to move the displayed boundaries based on the known speed of US propagation in the identified tissues. Various morphing techniques may be used to morph the image. According to various embodiments, morphing techniques include template or atlas based approach where a 3D shape reconstruction based on known a known template and/or atlas of the anatomy and/or structure of interest and available information from image data (original/non-corrected ultrasound images+correction information). Various embodiments, include feature based morphing may include a statistical relation between features (e.g., landmark locations) of a structure/anatomy is used to morph the 3D anatomy to the corrected state. Various embodiments, include a linear and/or non-linear spatial operations and interpolations for localized corrections.

An additional correction factor in accordance with the present disclosure includes identifying an actual tool position in an ultrasound image taken using 1540 m/s as the average speed of sound through tissue, comparing the actual tool position to a predicted position of the tool, and applying a correction factor based on the difference therebetween. For example, a tool is inserted within an anatomy, such as into the liver tissue 160, to a known depth, such as 15.5 mm. The area is then imaged using the US housing 16 based on an average speed of sound through the fat tissue 150 and the liver tissue 160 of 1540 m/s (see FIG. 3A, for example). Depth of the tool in the ultrasound image is compared to the known depth of the tool. The difference between the known depth and the imaged depth, such as about 3 mm, is then applied as a correction factor by the navigation processing unit 74 when tracking instruments, particularly at the depth of 15.5 mm.

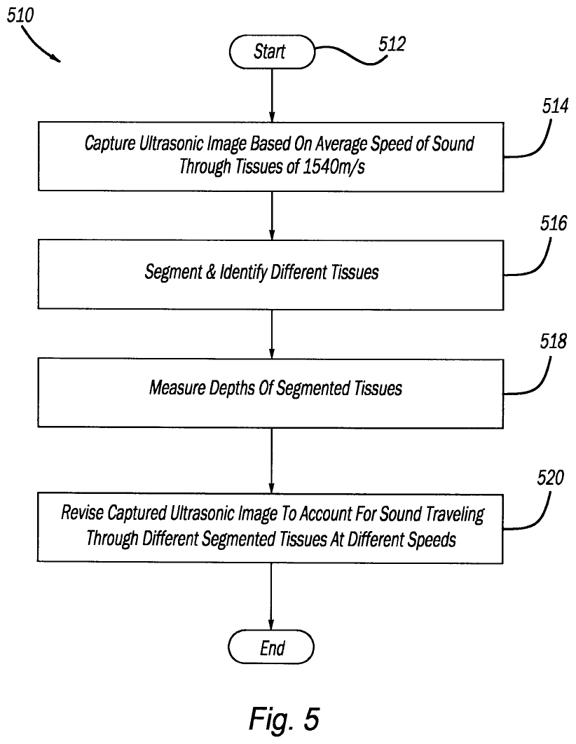

FIG. 5 illustrates an exemplary method 510 in accordance with the present disclosure of ultrasound depth calibration to improve navigational accuracy. The method 510 starts at block 512 and an ultrasound image data is captured in block 514. Further, an image may be generated in block 514 using a generally or averaged ultrasound (US) propagation speed of 1540 meters per second (m/s). At block 516 the different tissues imaged are segmented and identified. The tissues may be segmented and identified using any of the segmentation and identified procedures described above. This provides both the boundaries and the type of tissue imaged. The depths of the segmented tissues are next measured at block 518. At block 520, the ultrasonic image captured using 1540 m/s as the average speed of sound through all tissues is revised (e.g., morphed) to account for the different speeds that ultrasound travels or propagates through different segmented tissues. For example, at block 520 the image 110A is revised to image 110B, or the image 210A is revised or updated to the image 210B. The revised image 110B or 210B may then be used by the navigation processing unit 74 for enhanced tracking, particularly with respect to depth.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of calibrating an ultrasound imaging system, the method comprising:
capturing with the ultrasound imaging system ultrasound image data including a first tissue and a second tissue through which ultrasound waves travel at different speeds, the ultrasound image data captured based on a predetermined single speed of ultrasound waves through both the first tissue and the second tissue being the same, wherein a sonogram is based on the ultrasound image data;
segmenting the first tissue and the second tissue in the sonogram;
identifying a first depth of the first tissue and a second depth of the second tissue based on the sonogram;
identifying an actual first speed of ultrasound waves through the first tissue and an actual second speed of ultrasound waves through the second tissue; and
generating a calibrated image based on the sonogram that accounts for ultrasound waves traveling through the first tissue at the first actual speed that is different than the second actual speed of the ultrasound waves traveling through the second tissue.

2. The method of claim 1, wherein the segmenting includes visual inspection of the sonogram.

3. The method of claim 1, wherein the segmenting includes processing of the ultrasonic image by an image processing unit configured to identify the first tissue and the second tissue based on differences in texture.

4. The method of claim 1, wherein the segmenting includes identifying the first tissue and the second tissue based on a location of an ultrasound probe housing relative to anatomy.

5. The method of claim 1, wherein identifying the first depth and the second depth includes measuring the first tissue and measuring the second tissue on the ultrasonic image.

6. The method of claim 1, wherein identifying the first depth and the second depth includes estimating the first depth and the second depth based on patient parameters including one or more of age, sex, weight, and body mass index (BMI).

7. The method of claim 1, further comprising performing a surgical navigated procedure based on the calibrated image.

8. An ultrasound imaging system comprising:
an ultrasound housing including a transducer configured to emit and receive ultrasound waves;
an image processing unit configured to:
capture ultrasound image data including a first tissue and a second tissue through which ultrasound waves travel at different speeds, the ultrasound image data captured based on a predetermined single speed of ultrasound waves through both the first tissue and the second tissue being the same, wherein a sonogram is based on the ultrasound image data;
segment the first tissue and the second tissue in the sonogram;
identify the first tissue as a first tissue type and identify the second tissue as a second tissue type;
identify a first depth of the first tissue and a second depth of the second tissue based on the sonogram;
identify an actual first speed of ultrasound waves through the first tissue and an actual second speed of ultrasound waves through the second tissue; and
generate a calibrated image based on the sonogram that accounts for ultrasound waves traveling through the first tissue at the first actual speed that is different than the second actual speed of the ultrasound waves traveling through the second tissue.

9. The system of claim 8, wherein the segmenting includes processing of the ultrasonic image by an image processing unit configured to identify the first tissue and the second tissue based on differences in texture.

10. The system of claim 8, wherein the segmenting includes identifying the first tissue and the second tissue based on a location of an ultrasound probe housing relative to anatomy.

11. The system of claim 8, wherein identifying the first depth and the second depth includes measuring the first tissue and measuring the second tissue on the ultrasonic image.

12. The system of claim 8, wherein identifying the first depth and the second depth includes estimating the first depth and the second depth base on patient parameters including one or more of age, sex, weight, and body mass index (BMI).

13. The system of claim 8, further comprising performing a surgical navigated procedure based on the calibrated image.
